(19) United States
(12) Patent Application Publication
(10) Pub. No.: US 2006/0173398 A1
(43) Pub. Date: Aug. 3, 2006

(54) Method and apparatus for laparoscopic treatment of ascites

(76) Inventor: Jean P. Angelchik, Phoenix, AZ (US)

Correspondence Address: TOD R. NISSLE, P.C., P.O. Box 55630, Phoenix, AZ, 85078, US

(21) Appl. No.: 11/039,384

(22) Filed: Jan. 19, 2005

(51) Int. Cl. A61M 5/00

(52) U.S. Cl. 604/9

(57) ABSTRACT

A method and apparatus for removing ascitic liquid from the peritoneal cavity utilizes a valve which extends through the intestinal wall into the intestine. A seal is formed between the intestine and the valve to prevent the escape of material from the intestine into the peritoneal cavity and to prevent the valve from being drawn into the intestine.

This invention relates to a method and apparatus for removing ascitic fluid from the peritoneal cavity of a patient.

More particularly, the invention relates to a method and apparatus which removes ascitic liquid from the peritoneal cavity into a valve extending through the intestinal wall into the intestine and which effectively forms a seal between the intestine and the valve to prevent the escape of material from the intestine into the peritoneal cavity and to prevent the valve from being drawn into the intestine.

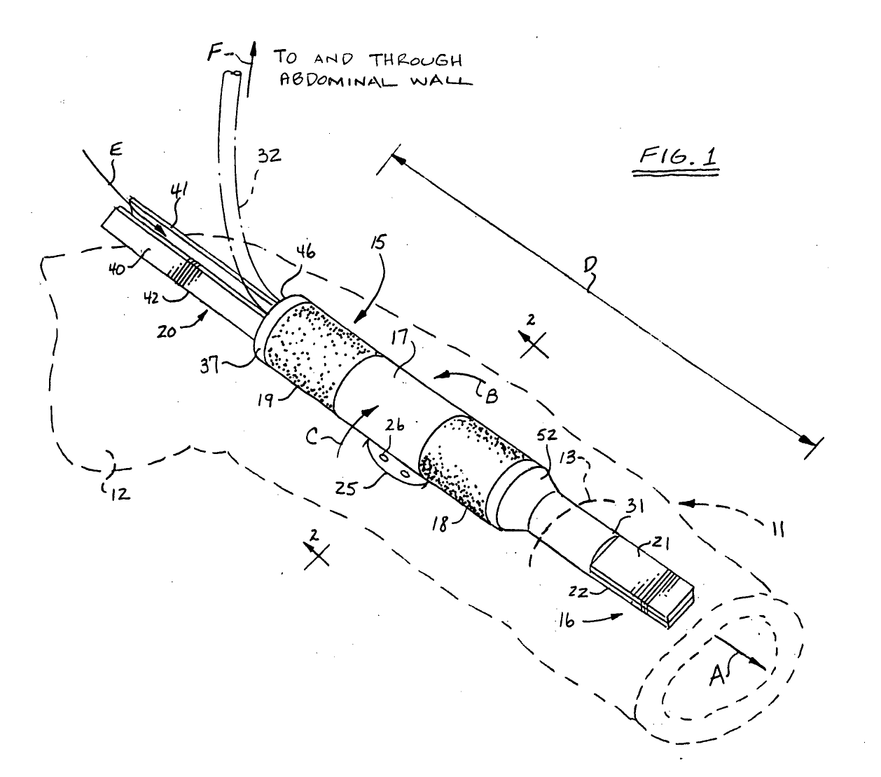

As recited in my U.S. Pat. No. 4,991,594, ascites is a serious medical condition characterized by the accumulation of body fluid in the peritoneal cavity. The accumulation of ascitic fluid can, unless corrected, contribute to the death of a patient. My U.S. Pat. No. 4,991,594 describes a method for draining ascitic fluid from the peritoneal cavity. The method typically requires that the intestine be severed to produce first (11A) and second (11B) open distal ends. A filter-valve (30) is attached to the first open distal end to permit ascitic fluid to drain into the intestine. The second open distal end (11B) is sutured to an opening (12) in the intestine (10A). See FIG. 1 of U.S. Pat. No. 4,991,594. While this method is acceptable, it requires major surgery. Also, while unlikely, if the sutures holding the filter-valve (30) to the first distal end (11A) work free, then the valve (30) can separate from the intestine, permitting infection of the peritoneal cavity.

Accordingly, it would be highly desirable to provide an improved method for draining ascitic fluid from the peritoneal cavity which would not require severing of the intestine and which would reduce the risk that the peritoneal cavity would be infected in the event a filter-valve separated from the intestine.

Therefore, it is a principal object of the invention to provide an improved method and apparatus for draining ascitic fluid from the peritoneal cavity, which method and apparatus can be used laproscopically.

A further object of the instant invention is to provide an improved method and apparatus which enables a valve for draining ascitic fluid to be attached to the intestine without severing the intestine.

Another object of the invention is to provide an improved method and apparatus for attaching an ascitic fluid-drainage valve to the intestine such that leakage from the intestine into the peritoneal cavity does not occur in the event the drainage valve separates from the intestine.

These and other, further and more specific objects and advantages of the invention will be apparent to those skilled in the art from the following detailed description thereof, taken in conjunction with the drawings, in which:

FIG. 1 is a perspective view illustrating a filter—valve constructed in accordance with the principles of the invention; and,

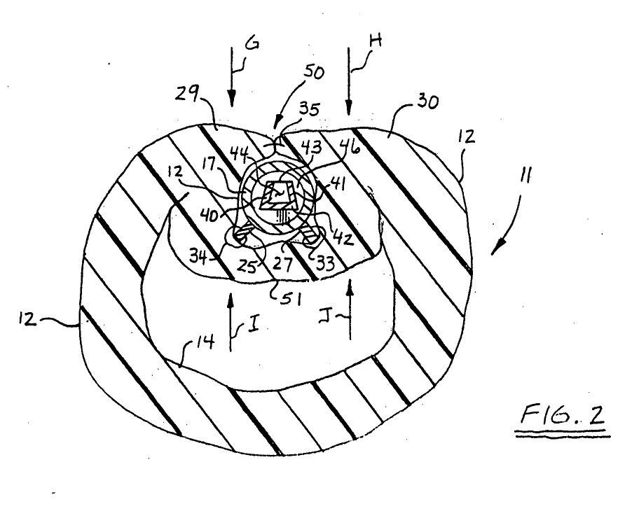

FIG. 2 is a section view of the valve of FIG. 1 illustrating further internal construction details thereof and illustrating the method of installing the valve in the intestine.

Briefly, in accordance with my invention, I provide a method for removing ascites liquid from the peritoneal cavity through the alimentary canal. The method includes the steps of making an incision through the wall of the intestine and positioning drain-valve means in the incision such that the drain-valve means extends from outside the intestine, through the incision, and into the intestine to permit ascitic fluid in the peritoneal cavity to flow through the drain-valve means into the intestine. Outer surface portions of the intestine are drawn over the drain-valve means to cover the incision such that the incision is sealed by the outer surface portions in the event the drain-valve means separates from the intestine. The outer surface portions are fixed in position over the incision.

In another embodiment of my invention, I provide a method for removing ascites liquid from the peritoneal cavity through the alimentary canal. The method includes the steps of making an incision through the wall of the intestine and positioning drain-valve means in the incision such that the drain-valve means extends from outside the intestine, through the incision, and into the intestine to permit ascitic fluid in the peritoneal cavity to flow through the drain-valve means into the intestine. The drain-valve means has an intermediate portion with a first end and a second end. A drain-valve is attached to the first end. A valve is attached to the second end. The first end includes an alloplast which permits fibroblastic growth to produce living cells in the interstices of the alloplast. Outer surface portions of the intestine are drawn over the drain-valve means to cover the incision such that the incision is sealed by the outer surface portions in the event the drain-valve means separates from the intestine. The outer surface portions are fixed in position over the incision.

In still another embodiment of my invention, I provide, in combination with an intestine in the peritoneal cavity, a valve for dispensing liquid into the intestine through an incision formed through the wall of the intestine. The valve includes an intermediate portion extending through the incision. The intermediate portion has a first end adjacent the intestine and a second end. The first end includes an alloplast which permits fibroblastic growth from the intestine to produce living cells in the interstices of the alloplast. A drain is connected to the first end of the intermediate portion and extends outwardly from the intestine into the peritoneal cavity. A valve is connected to the second end of the intermediate portion and extends into the intestine.

Turning now to the drawings, which depict the presently preferred embodiments of the invention for the purpose of illustrating the practice thereof, and not by way of limitation of the scope of the invention, and in which like reference characters refer to corresponding elements throughout the several views, FIGS. 1 and 2 illustrate a drain-valve unit 15 constructed in accordance with the principles of the invention and used in combination with the intestine 11 in the peritoneal cavity of an human or other animal. The unit 15 includes an intermediate portion 17 having a first end 37 and a second end 31. An alloplastic material 19 is wrapped around end 37. Alloplastic material 18 is also wrapped around portion 17 near end 31. The alloplastic material or cuffs 18, 19 can, by way of example and not limitation, comprise a dacron felt distributed Quinton Instrument Company of Seattle, Wash.; a collagen/silver matrix distributed by Davol Company of Cranston, R.I.; a dacron distributed by Davol Company of Cranston, Rhode, Island; a porous polytetrafluoroethylene (PROPLAST) distributed by Novamed Corporation of Houston, Tex.; GORETEX distributed by W. L. Gore and Associates, Inc. of Flagstaff, Ariz.; or, a porous polyurethane distributed by Thermedics, Inc. of Woburn, Mass.

A pair of fins 25 and 27 are attached to the bottom of and outwardly extend from the intermediate portion 17 of the filter-valve unit 15. Apertures 26 extend through each fin 25 and 27. Suture 33 and 34 is passed through apertures 26 and intestine 11 to affix unit 15 in place on intestine 11. Portions 29, 30 are drawn up and over the intermediate portion 17 of unit 15 and over incision 13 in intestine 11 in the manner described below.

Elongate pliable filter-drain 20 includes a pair of opposed, resilient flanges or flaps 40 and 41. Filter-drain floats and is free to move in the peritoneal cavity. Each flap 40, 41 is attached to elongate rectangular backing strip 42. Flaps 40, 41 and strip 42 partially encircle and define elongate channel 44. One end of filter-drain 20 is attached to circular panel 46 of end 37 and partially circumscribes an opening 43 formed through panel 46. Flaps 40 and 41 resiliently move inwardly and outwardly toward and away from channel 44. The space between the opposed distal ends of flaps 40 and 41, along with the resilient pliable nature of the flaps, helps prevent channel 44 from being completely blocked by chunks of fibrin or other material so that liquid cannot flow into channel 44 and through opening 43 in the manner indicated by arrow E in FIG. 1. Liquid flowing through opening 43 moves through hollow cylindrical intermediate portion 17 and then through one-way valve 16 into the inner area of intestine 11 circumscribed by wall 14. Valve 16 includes an opposed pair of pliable flaps 21, 22. Liquid can flow out through valve 16 in the direction of arrow A in FIG. 1, but cannot flow in the opposite direction back through valve 16 into portion 17. If desired, a plurality of spaced apart apertures 43 can be formed through panel 46 and a separate filter-drain 20 attached to each aperture 43 in the manner that filter-drain 20 in FIG. 1 is attached to aperture 43.

In operation, a small linear incision 13 is made in the intestine 11 and valve 21 and end 31 are inserted through incision 13 in the manner indicated in FIG. 1. In FIG. 1, valve 21 is positioned inside intestine 11 and end 31 is contacted by the portions of intestine 11 defining incision 13. The part of the intermediate portion 17 extending between end 31 and filter 20 is outside of intestine 11 adjacent outer surface 12. Suture 33, 34 is used to attach unit 15 to the outer surface 12 of intestine 11 in the manner indicated in FIG. 2. The outer surface of intestine 11 is drawn up and over unit 15 and incision 13 to form portions 29 and 30. The upper distal ends of portions 29 and 30 abut to form an elongate seam 50 which is generally parallel to the longitudinal axis of unit 15 and to arrow D in FIG. 1. Portions 29 and 30 are secured together along seam 50 by passing suture 35 through portions 29 and 30. Each portion 29, 30 is generally rectangular in shape and appearance and, as indicated by arrow D in FIG. 1, approximately extends from end 37 to end 31 of unit 15. This means that portions 29, 30 cover the part of intermediate portion 17 of unit 15 which extends out of incision 13 and over the outer surface 12 of intestine 11. Portions 29, 30 also extend over and completely cover incision 13 and cover the area of outer surface 12 which is immediately to the lower right of incision 13 in FIG. 1. This coverage of incision 13 by portions 29 and 30 is advantageous because in the event unit 15 somehow separates from intestine 11, portions 29, 30 cover and seal incision 13. Further, fibroblast from portions 29 and 30 grows into alloplasts 19 and 18 to form additional seals which anchor unit 15 in position on intestine 11 and prevent substances from leaking outwardly from within intestine 11 through seal 13. Which the fibroblasts may only adhere to the periphery of the alloplast, it is preferred that there is fibroblastic ingrowth in the alloplast such that fibroblast cells are formed in interstices in the alloplast. The peristaltic action of the intestine 11 facilitates the travel of fluid from the peritoneal cavity, through filter 20, through intermediate portion 17, and through valve 16 into intestine 11. Alloplast 18 is preferably bacteriostatic, as can be alloplast 19, due to its proximity to incision 13.

In an alternate embodiment of the invention, the filter-drain 20 is replaced with a feeding tube 32. Tube 32 extends through an opening in the abdominal wall to permit liquid nutrients, antibiotics, etc. to be fed into intestine 11 through tube 32 and unit 15. In this alternate embodiment of the invention, alloplasts 18, 19 can, if desired, be removed from unit 15 when the use of feeding tube 32 is temporary. Unit 15 can be installed using laparoscopic procedures. During such laparoscopic procedures, unit 15 need not be secured to intestine 11 with suture. This permits unit 15 to simply be pulled out when the feeding through tube 32 is completed. When unit 15 is pulled out of incision 13, portions 29 and 30 effectively seal incision 13 and prevent substances inside intestine 11 from traveling out through incision 13 into the peritoneal cavity. When unit 15 is utilized to withdraw ascitic fluid from the peritoneal cavity, it can also be installed laparoscopically. During the laparoscopic procedure several openings which are about five to ten millimeters wide are made through the abdominal wall. A small camera is installed through one opening. Unit 15, which is, at its largest dimension, only about five millimeters in diameter (portion 17), is inserted through another opening. Scalpels and other surgical instruments are inserted through the other openings and are, while the surgeon views the proceeding through the camera, used by a surgeon to position unit 15 on intestine 11 in the manner illustrated in FIGS. 1 and 2. The risk of infection and complications is, in comparison to making an incision through the abdominal wall which is sized to receive the surgeons fingers or hands, significantly reduced by using laparoscopic procedures.

In the presently preferred embodiment of unit 15, valve 16 is about four millimeters wide and nine millimeters long; end 31 is about six millimeters long; the outside diameter of the section of portion 17 carrying alloplasts 18 and 19 is about five millimeters. The overall length of unit 15 in FIG. 1 from the tip of valve 16 to end 37 is about fifty millimeters. The drain-filter 20 is about six to twelve inches long.

The pressure in the peritoneal cavity is greater than the pressure in the intestine 11, and along with the peristaltic action of the intestine 11, cause ascites fluid to flow from the peritoneal cavity, through drain 20, portion 17, and valve 16 into intestine 11. In fact, the forces acting cause fluid to flow from the peritoneal cavity into intestine 11 promote the induction of unit through incision 13 into intestine 11. As a result, the section of portion 17 circumscribed by alloplast 18 is larger than the diameter of end 31 which extends through incision 13 and, preferably but not necessarily, is larger than incision 13 such that the likelihood that portion 17 unit 15 will pass through incision 13 into intestine 11 is minimized. Similarly, alloplasts 18 and 19 serve to anchor unit 15 in place after fibroblastic ingrowth has occurred. It is important that the fibroblastic ingrowth penetrate the interstices of at least one of the alloplasts 18 and 19. Fibroblastic growth which merely adheres to the outer surface of alloplasts 18 or 19 does not provide a sufficient anchor because the fibroblastic growth can too readily be peeled off of and separated from the alloplast. If desired, to facilitate the sealing of incision 13, conical surface 52 can be abutted directly against incision 13. It is important that alloplast 18 not contact or extend into incision 13.

In FIG. 2, portions 29 and 30 are drawn over unit 15 and sutured together such that portion 17 of unit 15 is compressed between portions 29 and 30 and the portion 51 of intestine underlying unit 15 and/or portions 29 and 30. As a result, after portions 29 and 30 are sutured together they generate compressive forces acting in the direction of arrows G and H, while portion 51 generates forces acting in the direction of arrows I and J. Consequently, if unit 15 is then withdrawn from between portion 51 and portions 29 and 30, portion 29 moves toward portion 51 and portion 30 moves toward portion 51 to reduce and possibly eliminate the resulting spaces between portion 29 and portion 51 and between portion 30 and portion 51. Eliminating the space between portions 51 and portions 29, 30 functions to seal incision 13 to prevent material from escaping from intestine 11 into the peritoneal cavity.

Having described my invention in such terms as to enable those skilled in the art to understand and practice it, and having identified the presently preferred embodiments thereof, I claim:

1. A method for removing ascites liquid from the peritoneal cavity through the alimentary canal, said method comprising
(a) making an incision through the wall of the intestine;
(b) positioning drain-valve means in said incision such that said drain-valve means extends from outside said intestine, through said incision, and into said intestine to permit ascitic fluid in the peritoneal cavity to flow through said drain-valve means into said intestine;
(c) drawing outer surface portions of said intestine over said drain-valve means to cover said incision such that said incision is sealed by the outer surface portions in the event said drain-valve means separates from said intestine; and,
(d) fixing said outer surface portions in position over said incision.

2. A method for removing ascites liquid from the peritoneal cavity through the alimentary canal, said method comprising
(a) making an incision through the wall of the intestine;
(b) positioning in said incision a drain-valve means for draining ascites fluid into the alimentary canal, said drain-valve means having an intermediate portion with a first end and a second end, a drain attached to said first end, and a valve attached to said second end, said first end including an alloplast which permits fibroblastic growth to produce living cells in the interstices of said alloplast, said second end of said intermediate portion extending through said incision, said drain extending outwardly from said intestine into the peritoneal cavity, and said valve extending into said intestine such that ascitic fluid in the peritoneal cavity flows through said filter, said intermediate portion, and said valve into said intestine;
(c) drawing outer surface portions of said intestine over said drain-valve means to cover said incision and said intermediate portion such that said incision is sealed in the event said drain-valve means separates from said intestine; and,
(d) fixing said outer surface portions in position over said incision such that fibroblastic growth from said outer surface portions into said alloplast occurs.

3. In combination with an intestine in the peritoneal cavity, a valve for dispensing liquid into the intestine through an incision formed through the wall of the intestine, said valve including
(a) an intermediate portion extending through said incision and having a first end adjacent said intestine, a second end, said first end including an alloplast which permits fibroblastic growth from said intestine to produce living cells in the interstices of said alloplast;
(b) a drain connected to said first end of said intermediate portion and extending outwardly from said intestine into the peritoneal cavity;
(c) a valve connected to said second end of said intermediate portion and extending into said intestine.
